(19) United States
(12) Patent Application Publication
(10) Pub. No.: US 2025/0195525 A1
(43) Pub. Date: Jun. 19, 2025

(54) ANGIOTENSIN II RECEPTOR ANTAGONIST FOR THE PREVENTION OF SYSTEMIC DISEASES IN CATS

(71) Applicant: Boehringer Ingelheim Vetmedica GmbH, Ingelheim am Rhein (DE)

(72) Inventors: Silke HAAG-DIERGARTEN, Frankfurt am Main (DE); Karla DRECKMANN, Burgdorf (DE); Esther HERBERICH, Mainz (DE); Ingo Ulrich LANG, Ingelheim am Rhein (DE); Dagmar Maria POLOTZEK, Frankfurt am Main (DE); Christian TROETSCHEL, Waldalgesheim (DE); Philip WATSON, Wiesbaden (DE)

(21) Appl. No.: 18/978,301

(22) Filed: Dec. 12, 2024

(30) Foreign Application Priority Data
Dec. 15, 2023 (EP) 23216970.6

(51) Int. Cl. A61K 31/519; A61K 31/4178; A61K 31/4184; A61K 45/06; A61P 9/12

(52) U.S. Cl. CPC A61K 31/519; A61K 31/4178; A61K 31/4184; A61K 45/06; A61P 9/12

(57) ABSTRACT

A method is described for the prophylaxis of hypertension in a cat in need thereof. The method includes administration of an effective amount of angiotensin II receptor 1 (AT-1) antagonist (sartan) to the cat, wherein the prophylaxis of hypertension is a delay of onset of hypertension.

### FIELD OF THE INVENTION

The present invention relates to the field of veterinary medicine, especially to the prophylaxis of systemic diseases in cats. In particular, the present invention relates to a method for the prophylaxis of hypertension in a cat in need thereof, wherein the method comprises administration of an effective amount of angiotensin II receptor 1 (AT-1) antagonist (sartan) to the cat wherein the prophylaxis of hypertension is a delay of onset of hypertension.

### BACKGROUND OF THE INVENTION

Chronic Kidney Disease (CKD) as well as systemic hypertension are irreversible, progressive, and one of the most common medical reasons for medical evaluation of older cats. The prevalence of CKD increases with age and up to 35% of the geriatric cat population is affected. Clinical signs such as polyuria, polydipsia, lethargy, decreased appetite and weight loss often are described, and a considerable effect of CKD on the quality of life can be assumed. Furthermore, CKD negatively impacts survival, with an average life expectancy of 1-3 years once clinical signs become apparent. Target organ damage, such as systemic hypertension, plays an important role in disease progression. Systemic hypertension (HT) is defined as a persistent elevation of the systemic blood pressure. There are two subclassifications of HT; primary (or idiopathic) HT where no underlying disease can be identified, and secondary HT occurring as a complication of systemic disease.

Chronic renal failure (CRF), at least in its final stage is, regardless of the underlying causes, characterized by irreversible structural lesions of the kidney. Thereby, progressive irreversible lesions initially localized to one portion of the nephron (e.g., glomeruli, peritubular capillaries, tubules or interstitial tissue), are eventually responsible for the development of lesions in the remaining, but initially unaffected portions of nephrons due to their functional interdependencies. New nephrons cannot be formed to replace others irreversibly destroyed by disease. In a study of biopsy findings in cats with primary renal azotemia, tubulointerstitial nephritis was observed in 70%, glomerulonephropathy occurred in 15%, lymphoma in 11% and amyloidosis was observed in 2% of the samples. CRF is recognized by reduced kidney function or the presence of kidney damage (Polzin, Osborne, Ross 2005 in: Ettinger S J, Feldman C E (eds.) Textbook of Veterinary Internal Medicine, 6th, Vol 2. Chapter 260, 1756-1785).

One of the most common causes for secondary hypertension in cats is CKD. CKD related HT is believed to be induced by the activation of renin-angiotensin-aldosteron system (RAAS) and eventually aldosterone production. Effects are mainly mediated through the angiotensin-II receptor type 1 (AT-1). Chronic RAAS activation leads to persistent HT via systemic vasoconstriction, intravascular fluid expansion and sympathetic activation.

Regardless of the cause, HT can result in local or systemic disease via destructive effects to vascular beds of various organs. Clinically, HT related damage is frequently recognized in the eyes, brain, kidney, heart and the vessels. According to the ACVIM consensus panel on hypertension in dogs and cats injury to these organs resulting from persistent elevation of SBP is collectively termed target organ damage (TOD) (ACVIM Consensus Statement: Brown S, Atkins C, Carr A, Cowgill L, Davidson M, Egner B, Elliott J, Henik R, Labato M, Littman M, Polzin D, Ross L, Snyder P, and Stepien R. Guidelines for the Identification, Evaluation and Management of Systemic Hypertension in Dogs and Cats. J Vet Intern Med 2007; 21:542-558). Development of HT in cats with CKD leads to further progression of the disease.

Early management of both diseases can improve quality of life and prognosis. Compensatory chronic activation of the Renin-Angiotensin-Aldosterone System (RAAS) in CKD to maintain Glomerular Filtration Rate (GFR) increases Angiotensin-II (AT-II) production with renal concentrations exceeding plasma concentrations. Angiotensin-II is a central mediator of renal injury because of its ability to produce glomerular hypertension that results in glomerular damage, proteinuria, and activation of pro-inflammatory and profibrotic pathways.

Angiotensin II plays an important part in pathophysiology, particularly as the most potent agent for increasing blood pressure in humans. It is known that in addition to its effect of raising blood pressure Angiotensin II also has growth-promoting effects which contribute to left ventricular hypertrophy, vascular thickening, atherosclerosis, renal failure and stroke. In small animals, inhibition of the effects of Angiotensin II, via either ACE inhibitors have been shown to exhibit renoprotectiv effects through their simultaneous capacity to decrease blood pressure and control proteinuria.

Several AT-II receptors have been identified. The major detrimental renal effects of AT-II described above are mediated by AT1 receptors. The AT2 receptors modulate actions of AT-II that are renoprotective, namely vasodilation, natriuresis, inhibition of renin secretion, and anti-inflammatory, anti-ischemic and antifibrotic effects. The expression of the AT2 receptor is increased in pathologic circumstances. Angiotensin Converting Enzyme (ACE)-inhibitors prevent enzymatic conversion of AT-I, decreasing AT-II concentrations. However, in mammals, including the cat, alternative pathways (ACE-escape) exist for AT-II generation. Telmisartan is a non-peptide, AT II type-1 Receptor Antagonist (ARB) and selectively blocks the AT1 receptor with high affinity, while leaving beneficial effects of the AT2 receptor activation unaffected. Furthermore, the efficacy of ARBs is unaffected by ACE-escape mechanisms. The renoprotective properties of telmisartan have been demonstrated in ex vivo models and in vivo studies in human patients with diabetic and nondiabetic nephropathies. In contrast to other ARBs, telmisartan also is a partial agonist of the Peroxisome Proliferator-Activated Receptor Gamma (PPAR-y) receptor and its renoprotective properties are increased by this dual action. Also in cats there are data on the progression of CKD (Chakrabarti S, Syme H M, Elliott J. Clinicopathological Variables Predicting Progression of Azotemia in Cats with Chronic Kidney Disease. J Vet Intern Med 2012; 26 (2): 275-81; Sent U, Gössl R, Elliott J, Syme, HM, Zimmering T. Comparison of Efficacy of Longterm Oral Treatment with Telmisartan and Benazepril in Cats with Chronic Kidney Disease. J Vet Intern Med 2015; 29 (6): 1479-87).

Thus, one objective of the present invention consists in providing a new approach for the prophylaxis of cats against chronic kidney disease and hypertension. More specifically, one objective of the present invention consists in providing a new approach for the prophylaxis of cats against hypertension.

A furthermore general aspect of the present invention consists in providing a new therapeutic approach for the prophylaxis of cats against systemic diseases; preferably against systemic diseases which are related to Angiotensin II or associated with the renin-angiotensin-aldosterone system (RAAS). Another more general aspect of the present invention consists in providing a new therapeutic approach for the treatment and prophylaxis of cats against chronic kidney disease and hypertension.

Still a further aspect of the present invention consists in providing a new approach for the prophylaxis of hypertension in cats with a risk factor for hypertension, such as chronic kidney disease.

A more specific aspect relates to a new approach for the delay of onset of hypertension in cats with a risk factor for hypertension, such as chronic kidney disease.

### BRIEF DESCRIPTION OF THE DRAWING

FIG. 1 shows the progression of SBP over time comparing cats having received telmisartan or placebo.

### DETAILED DESCRIPTION OF THE INVENTION

Before the embodiments of the present invention, it shall be noted that as used herein and in the appended claims, the singular forms “a”, “an”, and “the” include plural reference unless the context clearly dictates otherwise. Thus, for example, reference to “a preparation” includes a plurality of such preparations, reference to the “carrier” is a reference to one or more carriers and equivalents thereof known to those skilled in the art, and so forth. Unless defined otherwise, all technical and scientific terms used herein have the same meanings as commonly understood by one of ordinary skill in the art to which this invention belongs. All given ranges and values may vary by 1 to 5% unless indicated otherwise or known otherwise by the person skilled in the art, therefore, the term “about” was omitted from the description. Although any methods and materials similar or equivalent to those described herein can be used in the practice or testing of the present invention, the preferred methods, devices, and materials are now described. All publications mentioned herein are incorporated herein by reference for the purpose of describing and disclosing the substances, excipients, carriers, and methodologies as reported in the publications which might be used in connection with the invention. Nothing herein is to be construed as an admission that the invention is not entitled to antedate such disclosure by virtue of prior invention.

The solution to the above technical problem is achieved by the description and the embodiments characterized in the claims.

In accordance with the present invention, methods are described herein for the prophylaxis of hypertension (also referred to as high blood pressure) in a cat in need thereof wherein the method comprises administration of an effective amount of angiotensin II receptor 1 (AT-1) antagonist (sartan) to the cat, wherein the cat has a risk factor for hypertension and the prophylaxis of hypertension is a delay of onset of hypertension.

One aspect of the present invention relates to an angiotensin II receptor 1 (AT-1) antagonist (sartan) for use in a method for the prophylaxis of hypertension in a cat in need of such prophylaxis, wherein the method comprises administration of an effective amount of angiotensin II receptor 1 (AT-1) antagonist (sartan) to the cat, wherein the prophylaxis of hypertension is a delay of onset of hypertension. A corresponding method of prevention of hypertension in a cat with an effective amount of angiotensin II receptor 1 (AT-1) antagonist (sartan), as well as the corresponding use of an angiotensin II receptor 1 (AT-1) antagonist (sartan) for the preparation of a medicament for treating a cat with a liquid pharmaceutical composition, are also intended to be comprised by the present invention.

In another aspect, the present invention relates to an angiotensin II receptor 1 (AT-1) antagonist (sartan) for use in a method for the delay of onset of hypertension in a cat, wherein the method comprises administration of an effective amount of angiotensin II receptor 1 (AT-1) antagonist (sartan) to the cat. A corresponding method for the delay of hypertension in a cat with an effective amount of angiotensin II receptor 1 (AT-1) antagonist (sartan), as well as the corresponding use of an angiotensin II receptor 1 (AT-1) antagonist (sartan) for the preparation of a medicament for treating a cat with a liquid pharmaceutical composition, are also intended to be comprised by the present invention.

In a specific embodiment of the present invention, the cat has a risk factor for hypertension. In a more specific embodiment, the present invention relates to methods for the prophylaxis of hypertension in a cat in need of such prophylaxis, wherein the method comprises administration of an effective amount of angiotensin II receptor 1 (AT-1) antagonist (sartan) to the cat, wherein the prophylaxis of hypertension is a delay of onset of hypertension and wherein the cat has a risk factor for hypertension. In a preferred embodiment, the risk factor is CKD.

In particular, the following items are disclosed herein:

1. An angiotensin II receptor 1 (AT-1) antagonist (sartan) for use in a method for the prophylaxis of hypertension in a cat in need of such prophylaxis, wherein the method comprises administration of an effective amount of angiotensin II receptor 1 (AT-1) antagonist (sartan) to the cat, wherein the prophylaxis of hypertension is a delay of onset of hypertension.

2. An angiotensin II receptor 1 (AT-1) antagonist (sartan) for use in a method for the delay of onset of hypertension in a cat, wherein the method comprises administration of an effective amount of angiotensin II receptor 1 (AT-1) antagonist (sartan) to the cat.

3. The angiotensin II receptor 1 (AT-1) antagonist (sartan) for use in a method according to item 1 or 2, wherein the cat has a risk factor for hypertension, preferably Chronic Kidney Disease (CKD).

4. The angiotensin II receptor 1 (AT-1) antagonist (sartan) for use in a method according to item 3, wherein the risk factor for hypertension is selected from one or more of the following:
A systolic blood pressure (SBP) between 120 mmHg and 140 mmHg, or between 120 mmHg and 160 mmHg, or between 140 mmHg and 160 mmHg;
Age of the cat;
Hyperthyroidism; or
Chronic Kidney Disease (CKD), wherein CKD is preferably characterized by a creatinine value of more than 1.9 mg/dl (168 μmol/L) and less than 5.0 mg/dl (440 μmol/L); and/or
A Urine Specific Gravity (USG) of more than 1.035 in the cat.

5. The angiotensin II receptor 1 (AT-1) antagonist (sartan) for use in a method according to one of the preceding items, wherein the delay of onset of hypertension is associated with a slowing down of disease progression of CKD or hyperthyroidism or a delay of onset of systemic hypertension.

6. The angiotensin II receptor 1 (AT-1) antagonist (sartan) for use in a method according to one of the preceding items, wherein the delay of onset of hypertension is a delay of the time after which a systolic blood pressure of 160 mmHg is reached compared to cats having not received a sartan.

7. The angiotensin II receptor 1 (AT-1) antagonist (sartan) for use in a method according to one of the preceding items, wherein the hypertension is associated with chronic kidney disease or hyperthyroidism.

8. The angiotensin II receptor 1 (AT-1) antagonist (sartan) for use in a method according to one of the preceding items, wherein the hypertension is idiopathic hypertension.

9. The angiotensin II receptor 1 (AT-1) antagonist (sartan) for use in a method according to one of the preceding items, wherein the delay of onset of hypertension is at least 10 days, or at least 20 days, or at least 30 days, or at least 40 days, or at least 50 days, or at least 60 days, or at least 70 days, or at least 80 days compared to cats having not received a sartan.

10. The angiotensin II receptor 1 (AT-1) antagonist (sartan) for use in a method according to one of the preceding items, wherein the angiotensin II receptor 1 (AT-1) antagonist is selected from the group consisting of: candesartan, eprosartan, irbesartan, losartan, tasosartan, telmisartan, valsartan, and a pharmaceutically acceptable salt of said angiotensin II receptor agonist, preferably telmisartan or a pharmaceutically acceptable salt thereof.

11. The angiotensin II receptor 1 (AT-1) antagonist (sartan) for use in a method according to one of the preceding items, wherein the effective amount of the angiotensin II receptor 1 (AT-1) antagonist is in a sub-therapeutic range.

12. The angiotensin II receptor 1 (AT-1) antagonist (sartan) for use in a method according to one of the preceding items, wherein the effective amount of the angiotensin II receptor 1 (AT-1) antagonist administered by oral route is 0.50 to 1.0 mg angiotensin II receptor 1 (AT-1) antagonist per kg of body weight.

13. The angiotensin II receptor 1 (AT-1) antagonist (sartan) for use in a method according to one of the preceding items, wherein the effective amount of the angiotensin II receptor 1 (AT-1) antagonist administered by oral route is about 1.0 mg angiotensin II receptor 1 (AT-1) antagonist per kg of body weight.

14. The angiotensin II receptor 1 (AT-1) antagonist (sartan) for use in a method according to one of the preceding items, wherein the effective amount of the sartan is administered in a daily dosage amount that is varied over an administration period, the daily dosage amount of the sartan for a first period of time during the administration period is 0.50 to 1.0 mg/kg of body weight, and the daily dosage amount of the sartan is decreased for a second period of time subsequent the first period of time during the administration period.

15. The angiotensin II receptor 1 (AT-1) antagonist (sartan) for use in a method according to one of the preceding items, wherein the daily dosage amount is varied based upon a systolic blood pressure (SBP) value measured for the cat.

16. The angiotensin II receptor 1 (AT-1) antagonist (sartan) for use in a method according to one of the preceding items, wherein the daily dosage amount of the sartan is decreased for the second period of time by an incremental amount ranging from 0.10 to 0.50 mg/kg of body weight.

17. The angiotensin II receptor 1 (AT-1) antagonist (sartan) for use in a method according to one of the preceding items, wherein the pharmaceutical composition comprising the effective amount of angiotensin II receptor 1 (AT-1) antagonist is administered together with at least one other drug to a cat in need thereof.

18. The angiotensin II receptor 1 (AT-1) antagonist (sartan) for use in a method according to one of the preceding items, wherein the other drug is selected from the group consisting of Ca-channel blockers (e.g. Amlodipine), beta-blockers (e.g. Atenolol, Carvediol), cardiotonic-Ca-sensitising agents (e.g. Pimobendan, Levosimendan), selective If-current inhibitors (i.e. Cilobradine, Ivabradine), ACE inhibitors (e.g. ramipril, benazepril, enalapril); anti-obesity drugs (such as Amphetamine derivatives, Sibutramine, Orlistat, Rimonabat).

19. A method for the delay of onset of hypertension in a cat, wherein the method comprises administration of an effective amount of angiotensin II receptor 1 (AT-1) antagonist (sartan) to a cat.

20. A method for the delay of onset of hypertension in a cat with CKD, wherein the method comprises administration of an effective amount of angiotensin II receptor 1 (AT-1) antagonist (sartan) to a cat.

21. A method for the prophylaxis of hypertension in a cat, wherein the method comprises administration of an effective amount of angiotensin II receptor 1 (AT-1) antagonist (sartan) to a cat and the prophylaxis of hypertension is a delay of onset of hypertension.

22. The method according to one of items 19 to 21, comprising

Determination, if the cat has a risk factor for hypertension selected from one or more of the following:
A systolic blood pressure (SBP) between 120 mmHg and 140 mmHg, or between 120 mmHg and 160 mmHg, or between 140 mmHg and 160 mmHg;
Age of the cat;
Hyperthyroidism; or
Chronic Kidney Disease (CKD), wherein CKD is preferably characterized by a creatinine value of more than 1.9 mg/dl (168 μmol/L) and less than 5.0 mg/dl (440 μmol/L); and/or
A Urine Specific Gravity (USG) of more than 1.035; and
Administration of an effective amount of angiotensin II receptor 1 (AT-1) antagonist (sartan) to the cat with a risk factor for hypertension.

23. The method according to any one of items 19 to 22, wherein the onset of hypertension is associated with progression of chronic kidney disease or hyperthyroidism.

24. The method according to any one of items 19 to 23, wherein the hypertension is idiopathic hypertension.

25. The method according to any one of items 19 to 24, wherein the angiotensin II receptor 1 (AT-1) antagonist is selected from the group consisting of: candesartan, eprosartan, irbesartan, losartan, tasosartan, telmisartan, valsartan, and a pharmaceutically acceptable salt of said angiotensin II receptor agonist.

26. The method according to any one of items 19 to 25, wherein the angiotensin II receptor 1 (AT-1) antagonist is administered in a sub-therapeutic range.

27. The method according to any one of items 19 to 26, wherein the angiotensin II receptor 1 (AT-1) antagonist is administered by oral route in a dosage of 0.50 to 1.0 mg/kg of body weight.

28. The method according to any one of items 19 to 27, wherein the angiotensin II receptor 1 (AT-1) antagonist is administered by oral route in a dosage of about 1.0 mg/kg of body weight.

29. The method according to any one of items 19 to 28, wherein the method further comprises administration of at least one other drug to the cat in need thereof.

30. The method according to item 29, wherein the at least one other drug is selected from the group consisting of Ca-channel blockers (e.g. Amlodipine), beta-blockers (e.g. Atenolol, Carvediol), cardiotonic-Ca-sensitising agents (e.g. Pimobendan, Levosimendan), selective If-current inhibitors (i.e. Cilobradine, Ivabradine), ACE inhibitors (e.g. ramipril, benazepril, enalapril); anti-obesity drugs (such as Amphetamine derivatives, Sibutramine, Orlistat, Rimonabat).

The use of angiotensin II receptor 1 antagonists (sartans) in cats for an indication is known. Blockage of angiotensin II receptor 1 is a treatment concept which differs from blockage of angiotensin converting enzyme as known from the ACE-inhibitors. Receptor blockage is more specific and complete and further downstream in the physiologic cascade of the RAAS system. The present invention is based on certain unexpected findings, namely, that a dosage of sartan for prophylaxis of hypertension in a cat can be down titrated or decreased over an administration period, particularly when the response in the cat is determined to be positive from such administration (e.g., a systolic blood pressure of the cat is reduced as a result of such administration and is further maintained at or below a certain level even with a reduction in dosage amount of the sartan being administered).

In studies, it has been found that cats tolerate a pharmacodynamically effective dose of sartans. For example, in an open-label study in nondiabetic, hypertensive human patients with proteinuric nephropathies the effects on renal outcome of low (80 mg once daily) and high dose (80 mg twice daily) telmisartan were compared. The results reinforced the concept that more effective RAAS inhibition achieved by a high dose of 160 mg daily. This dose corresponds to a plasma level of about 2800±2400 ng/ml (Cmax±SD), which exceeds the no-effect doses in toxicities studies in animals such as dogs and rats. (Investigator brochure 1994, data on file) The resulting dose of about 2 to 3 mg/kg body weight and day was thus expected to be toxic in cats. Pilot-toxicity studies have surprisingly shown that such a dose (up to 3 mg/kg) is well tolerable in cats. As used herein, the term “mg/kg” refers to dosage amount in mg per kg bodyweight of the cat.

Moreover, it was found that sartans effectively block the angiotensin II receptor 1 also in cats. This finding was unexpected, as the absolute bioavailability in cats is very low and the mean residence time and plasma half life are rather short in cats as compared to human beings. The oral bioavailability was calculated to 33.6% as compared to human beings. The mean tₘₐₓ oral was 0.44 hours and the Cₘₐₓ oral was 138.1 ng/ml. The mean t_{1/2} oral was 2.17 hours. The mean AUC→˜ oral was calculated to 150 (ng×h/ml), and the mean V/f oral was 20.4 l/kg. The mean AUC→˜ intravenous was calculated to 385 (ng×h/ml). The mean t_{1/2} intravenous was 2.25 hours and the mean V/f oral was 8.8 l/kg. From this information, which was newly generated, it can be concluded that sartans, preferably telmisartan, can be used in cats with systemic diseases, such as chronic kidney disease, such as for example chronic renal failure, including chronic renal insufficiency.

TABLE 1
Abbreviations

| Abbreviation | Pharmacokinetic parameters |
|---|---|
| AUC | area under the plasma concentration time |
| | curve |
| C max | maximum measured plasma concentration |
| V/f | Volume of distribution (V) whereas f is the |
| | absolute bioavailability |
| MRT | mean residence time |
| t ½ | terminal half-live |
| t max | time to reach Cmax |

Thus, in an embodiment, the present invention relates to a method for the prophylaxis of hypertension in a cat in need of such prophylaxis, wherein the method comprises administration of an effective amount of angiotensin II receptor 1 (AT-1) antagonist (sartan) to the cat, wherein the cat has a risk factor for hypertension and the prophylaxis of hypertension is a delay of onset of hypertension. The risk factor for hypertension is selected from one or more of the following:
A systolic blood pressure (SBP) between 120 mmHg and 140 mmHg, or between 120 mmHg and 160 mmHg, or between 140 mmHg and 160 mmHg;
Age of the cat;
Hyperthyroidism; or
Chronic Kidney Disease (CKD),
wherein CKD is preferably characterized by a creatinine value of more than 1.9 mg/dl (168 μmol/L) and less than 5.0 mg/dl (440 μmol/L); and/or
A Urine Specific Gravity (USG) of more than 1.035 in the cat.
   In a preferred embodiment, the present invention relates to a method for the prophylaxis of hypertension in a cat in need of such prophylaxis, wherein the method comprises administration of an effective amount of angiotensin II receptor 1 (AT-1) antagonist (sartan) to the cat, wherein the cat has CKD and the prophylaxis of hypertension is a delay of onset of hypertension.

According to one aspect of the present invention, those parameters are used to determine if a cat has a risk factor for hypertension and shall include cats, which are in a non-hypertensive (normotensive) or pre-hypertensive state (referring to table 2-2). More specifically, this includes cats which have a systolic blood pressure of less than 160 mmHg, preferably between 120 mmHg and 140 mmHg, or between 140 mmHg and 160 mmHg or between 130 mmHg and 160 mmHg or more preferably between 140 mmHg and 160 mmHg or less than 180 mmHg or less than 200 mmHg, more specifically between 120 mmHg and 180 mmHg or between 130 mmHg and 180 mmHg or between 140 mmHg and 180 mmHg or between 160 mmHg and 180 mmHg or between 120 mmHg and 200 mmHg or between 130 mmHg and 200 mmHg or between 140 mmHg and 200 mmHg or between 160 mmHg and 200 mmHg or between 180 mmHg and 200 mmHg.

Prevalence of hypertension increases with age and is commonly observed in cats above 5 or 7 years of age. Therefore, the age of the cat is a risk factor for hypertension. In a preferred embodiment, the risk factor is an age of the cat of more than 5 years, or more than 6 years, or preferably more than 7 years, or more than 8 years, or more preferably more than 9 years, or more than 10 years, or more than 11 years, or more than 12 years, or more than 13 years, or more than 14 years, or more than 15 years, or more than 16 years, or more than 17 years, or more than 18 years, or between 5 and 25 years, or between 6 and 25 years, or between 7 and 25 years, or between 8 and 25 years, or between 9 and 25 years, or between 10 and 25 years, or between 11 and 25 years, or between 12 and 25 years, or between 13 and 25 years, or between 14 and 25 years, or between 15 and 25 years, or between 16 and 25 years, or between 17 and 25 years, or between 18 and 25 years, or between 19 and 25 years, or between 20 and 25 years.

Major risk factors are hyperthyroidism or Chronic Kidney Disease. In a preferred configuration, the CKD is characterized by specific creatinine values and Urine Specific Gravity. The determination of the creatinine value and the Urine Specific Gravity (USG) shall include cats with newly diagnosed CKD and a creatinine value of more than 1.9 mg/dl (168 μmol/L) and less than 5.0 mg/dl (440 μmol/L) and a Urine Specific Gravity (USG) of more than 1.035.

According to the present invention, the prophylaxis of hypertension is a delay of onset of hypertension. This means that the progression of hypertension is delayed. In a more specific aspect of the present invention, the delay of onset of hypertension is a slowing down of disease progression of CKD or hyperthyroidism or a delay of onset of systemic hypertension. More specifically, the delay of onset of hypertension is a delay of the time after which a systolic blood pressure of at least 160 mmHg is reached compared to cats having not received a sartan.

In a preferred embodiment of the present invention, the delay of onset of hypertension is at least 10 days, or at least 20 days, or preferably at least 30 days, or at least 40 days, or more preferably at least 50 days, or at least 60 days, or even more preferably at least 70 days, or at least 80 days, or between 1 and 10 days, or between 1 and 20 days or between 1 and 30 days, or between 1 and 40 days, or between 1 and 50 days, or between 1 and 60 days, or between 1 and 70 days, or between 1 and 80 days, or between 10 and 20 days, or between 10 and 30 days, or between 10 and 40 days, or between 10 and 50 days, or between 10 and 60 days, or between 10 and 70 days, or between 10 and 80 days, or between 20 and 30 days, or between 20 and 40 days, or between 20 and 50 days, or between 20 and 60 days, or between 20 and 70 days, or between 20 and 80 days, or between 30 and 40 days, or between 30 and 50 days, or between 30 and 60 days, or between 30 and 70 days, or between 30 and 80 days, or between 40 and 50 days, or between 40 and 60 days, or between 40 and 70 days, or between 40 and 80 days, or between 50 and 60 days, or between 50 and 70 days, or between 50 and 80 days, or between 60 and 70 days, or between 60 and 80 days, or between 70 and 80 days compared to cats having not received a sartan.

The term “systemic disease”, as used herein, means but is not limited to cardiovascular such as dilated cardiomyopathy (DCM), mitral valve insufficiency (MI), hypertrophic cardiomyopathy (HCM); and other acquired or hereditary heart diseases, e.g. cardiopulmonary diseases, systemic hypertension for example hypertension associated with renal diseases, chronic renal failure and other vascular diseases, or metabolic disorders such as diabetes mellitus. Thus, according to another aspect, the present invention relates to a method for the prophylaxis of a systemic disease in cats by the administration of an effective amount of said angiotensin II receptor 1 (AT-1) antagonist (sartan) to that cat, wherein the systemic disease is selected from the group of cardiovascular diseases, such as dilated cardiomyopathy (DCM), mitral valve insufficiency (MI), hypertrophic cardiomyopathy (HCM) and other acquired or hereditary heart diseases, hypertension (including systemic hypertension), metabolic disorders like diabetes mellitus.

The term “systemic hypertension”, as used herein, means but is not limited to, forms of hypertension associated with renal diseases, chronic renal failure and other vascular diseases. For example, systemic hypertension can include hypertension of an unknown cause, hypertension associated with chronic kidney disease, hypertension associated with hyperthyroidism, controlled hypertension and idiopathic hypertension. In particular, systemic hypertension (also referred to herein as hypertension) is defined as a persistent elevation of the systemic blood pressure (e.g., having a Systolic Blood Pressure or SBP of 160 mmHg or greater). There are two subclassifications of hypertension: primary (or idiopathic) hypertension in which no underlying disease can be identified, and secondary hypertension occurring as a complication of a systemic disease. In recent years, idiopathic hypertension has been increasingly recognized in small animal medicine. The most common causes for secondary hypertension in cats are chronic kidney disease (CKD) and hyperthyroidism. Other diseases such as hyperaldosteronism or pheochromocytoma are very rare causes for secondary hypertension in cats and usually result in severe and often therapy resistant hypertension.

The pathogenesis of idiopathic hypertension in feline patients is not fully understood. However, activation of renin-angiotensin-aldosteron system (RAAS) is thought to be present in affected cats. Chronic kidney disease related HT is believed to be induced by the activation of RAAS and eventually aldosterone production. Effects are mainly mediated through the angiotensin-II receptor type 1 (AT-1). Chronic RAAS activation leads to persistent hypertension via systemic vasoconstriction, intravascular fluid expansion and sympathetic activation. The underlying mechanism of hypertension secondary to feline hyperthyroidism remains undetermined, although dysfunction of RAAS is suspected. In some cats, hypertension is present at the time of diagnosis of hyperthyroidism, while in others hypertension develops after euthyroid state has been restored.

Regardless of the cause, hypertension can result in local or systemic disease via destructive effects to vascular beds of various organs. Clinically, hypertension related damage is frequently recognized in the eyes, brain, kidney, heart and the vessels. According to the ACVIM consensus panel on hypertension in dogs and cats (Brown et al. Guidelines for the identification, evaluation, and management of systemic hypertension in dogs and cats. ACVIM Consensus Statement. J Vet Intern Med 2007; 21:542-558), injury to these organs resulting from persistent elevation of systolic blood pressure (SBP) is collectively termed target organ damage (TOD), where TOD refers to a continuum of reversible to irreversible injury and relatively mild to potentially life-threatening consequences. Hypertension in cats possesses a significant risk for further organ damage. Activation and/or dysfunction of RAAS appears to play a crucial role in the development of feline hypertension of various causes. Consequently, RAAS inhibition seems a reasonable therapeutic target for hypertensive cats with either idiopathic hypertension, or secondary hypertension due to CKD or hyperthyroidism.

In humans, angiotensin II receptor 1 (AT1 receptor antagonists (sartans) are known to significantly reduce proteinuria in both diabetic and non-diabetic patients, even in those with mild to moderate chronic renal failure (CRF). Moreover, there is published evidence for the efficacious use of AT1 receptor antagonists for treatment of nephropathies in type II diabetes (Cupisti A, et al., 2003, Biomed Pharmacother; 57 (3-4): 169-172; Rysava R, et al., 2005, Press Monit; (10 (4): 207-213; WO92/10182). In cats tubulointerstitial nephritis is reported to be the major causative (>70%) finding for CRF whereas in human beings and dogs glomerulonephropathy is more prominent compared to cats. Glomerular lesions are more often seen in dogs and humans and consequently the clinical finding of moderate to marked proteinuria, resulting from loss of glomerular permselectivity, is more common in dogs and humans. Tubulointerstitial nephritis as seen in cats showed less proteinuria. Proteinuria is recognized as an important predictor of disease progression in humans and dogs with spontaneous kidney disease and reduction of proteinuria is associated with improved outcome in clinical trials to show the renoprotective effects of blocking the RAAS by either ACE or ARBs in human suffering from nephropathy (Karalliede & Viberti, J Human Hypertension 2006). Due to the fact that there is less proteinuria in cats because of the tubulointerstitial origin of CRF, the reduction of proteinuria as renoprotective effect in delaying progression of CRF might be expected to be less important in this species. However, in a clinical field trial an independent and significant correlation between proteinuria (determined as UPC) and survival in cats suffering from CRF have been reported. Surprisingly, even in azotemic cats with only minor proteinuria (acc. to IRIS, UPC <0.25) this correlation was evident (Syme, Elliot 2006, J Vet Intern Med, 20, 528-535).

Thus, according to a preferred embodiment the systemic disease is chronic kidney disease, preferably chronic renal failure, e.g. as defined as stage II to IV in Table 2-1.

The diagnosis of reduced kidney function such as chronic renal failure is based upon exclusion of pre- and postrenal causes and standard blood markers, e.g. urea and creatinine in plasma or serum. Abnormal concentrations of these parameters are described as azotemia. Standard urine markers of reduced kidney function include urine specific gravity, proteinuria and others (Polzin D J, Osborne C A, Ross S, 2005: Chronic Kidney Disease, In: Ettinger S J, Feldman E C (ed.) Textbook of Veterinary Internal Medicine 6th edition, W.B. Saunders Company, Philadelphia, USA). The international renal interest society (IRIS) has proposed a staging system based on azotemia to define CRF patients (Polzin D J, 2006: Treating feline kidney disease: an evidence-based approach, Proceedings of The North American Veterinary Conference). The main category for staging being plasma creatinine [mg/dl], which is completed by two subcategories independent from stage, urine protein: creatinine ratio (UPC) and blood pressure [mmHg]. With the applied system, feline patients are staged along a continuum of progressive kidney disease.

TABLE 2-1
Stages of feline chronic kidney disease

| | | | | Subcategory systolic |
|---|---|---|---|---|
| | Plasma | | Subcategory UPC | blood pressure |
| | creatinine | | (independent from | (mmHg, independent |
| Stage | (mg/dl) | Comments | stage) | from stage) |
| I | <1.6 | Non-azotemic: some | <0.2 = Non- | <150 = minimal risk |
| | | other renal abnormality | proteinuric | of end-organ damage |
| | | is present | | |
| II | 1.6-2.8 | Mildly azotemic: | | 150-159 = low risk of |
| | | usually mild clinical | | end-organ damage |
| | | signs | | |
| III | 2.9-5.0 | Moderately azotemic: | 0.2-0.4 = | 160-179 = moderate |
| | | many extra-renal | Borderline | risk of end-organ |
| | | clinical signs | proteinuric | damage |
| IV | >5.0 | Severely azotemic: | >0.4 = Proteinuric | ≥180 = high risk of |
| | | invasive life support | | end-organ damage |
| | | methods required | | |

TABLE 2-2
Ranges of systolic blood pressure (SBP) in cats

| Systolic blood pressure (mm Hg) | Substage of SBP |
|---|---|
| <140 | Normotensive |
| 140-159 | Prehypertensive |
| 160-179 | Hypertensive |
| ≥180 | Severely hypertensive |

As described herein, a method for the prophylaxis of chronic renal failure in cats comprises administration of an effective amount of angiotensin II receptor 1 (AT-1) antagonist (sartan) to that cat in need of such prophylaxis and wherein said chronic renal failure is characterized by any one of the clinical manifestations as listed in table 2-1, or any combination thereof. For example, the present invention relates to a method for the prophylaxis of cats having a plasma creatine of ≥1.6 (mg/dl of blood), and/or having a proteinuric of ≥0.2 (subcategory UPC), wherein the method comprises administration of an effective amount of angiotensin II receptor 1 (AT-1) antagonist (sartan) to that cat in need thereof.

A comprehensive list of angiotensin II receptor antagonists can be found on pages 2 to 22 of WO 92/10182 and pages 7 to 18 of WO 95/26188, which all are incorporated herein by reference. Angiotensin II receptor antagonists are described inter alia in EP-A-253310, EP-A-323841, EP-A-324377, EP-A-420237, EP-A-443983, EP-A-459136, EP-A-475206, EP-A-502314, EP-A-504888, EP-A-514198, WO 91/14679, WO 93/20816, WO 02/092081, U.S. Pat. Nos. 4,355,040, 4,880,804 and 6,028,091. Forms which are frequently mentioned are sartans, such as candesartan, eprosartan, irbesartan, losartan, olmesartan, tasosartan, telmisartan or valsartan. Those which are particularly preferred according to the present invention are irbesartan, losartan und telmisartan. All of these sartans, or pharmaceutical salts or polymorphs thereof are well known to a person skilled in the art, and its use is within the meaning of the present invention.

Thus the present invention relates to a method for the prophylaxis of cats suffering from hypertension, wherein the method comprising administration of an effective amount of angiotensin II receptor 1 (AT-1) antagonist (sartan) to that cat in need of such a prophylaxis and wherein the angiotensin II receptor 1 (AT-1) antagonist (sartan) is selected from the group consisting of: candesartan, eprosartan, irbesartan, losartan, olmesartan, tasosartan, telmisartan or valsartan, preferably of irbesartan, losartan und telmisartan.

Telmisartan is an angiotensin II receptor antagonist developed for the treatment of hypertension and other medical indications as disclosed in EP-A-502314. Its chemical name is 4′-[2-n-propyl-4-methyl-6-(1-methylbenzimidazol-2-yl)-benzimidazol-1-ylmethyl]-biphenyl-2-carboxylic acid having the following structure:

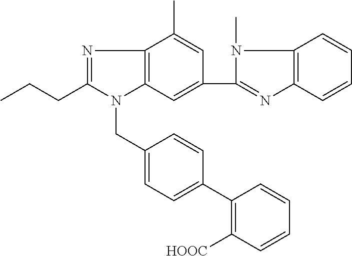 [SMILES: CCCc1nc2c(C)cc(-c3nc4ccccc4n3C)cc2n1Cc1ccc(-c2ccccc2C(=O)O)cc1]

Telmisartan is already sold on the market under the trade name Micardis® (Boehringer Ingelheim, Germany) for treatment/prophylaxis of humans. Telmisartan is also licensed in the EU under the trade name Semintra® for the reduction of proteinuria associated with CKD in cats. It exists in two polymorphic forms as disclosed in WO 00/43370, U.S. Pat. Nos. 6,358,986 and 6,410,742. Sodium salts of telmisartan and its solvate, hydrate, and hemihydrate are disclosed in WO 03/037876.

Thus, according to a further embodiment, the present invention relates to a method for the prophylaxis of a hypertension in cats, wherein the method comprising administration of an effective amount of telmisartan or pharmaceutically acceptable salt thereof, preferably of a telmisartan as mentioned above.

As already mentioned herein, it has been surprisingly found that use of telmisartan effectively inhibits the angiotensin II receptor pressure response in cats. Moreover, it has been found that doses of less than 0.05 mg telmisartan per kg body weight of cat led to an inhibition of the blood pressure response of about 75% in the majority of the tested cats. Moreover, a study in laboratory cats was established to investigate the Angiotensin II induced increase in diastolic blood pressure prior to and after administration of telmisartan. This test was established to estimate the potency as well as the duration of action of sartans, in particular of telmisartan in cats. Approximately 24 hours after the last oral dosing, diastolic blood pressure responses to increasing intravenous doses of Angiotensin II were significantly reduced when the target dose of telmisartan was compared with Placebo. Thus it could be concluded that administration of the target dose, despite the short elimination half-life and bioavailability, in the cat given once daily is capable to exhibit the intended pharmacodynamic action and duration.

For the treatment of hypertension in cats using sartans such as telmisartan, it has been described that for a first period of time during the administration the daily dosage amount of the sartan is 1.0 to 5.0 mg/kg of body weight to achieve a reduction of blood pressure in cats. In specific examples, the sartan telmisartan is administered in liquid form at a daily dosage amount of 2.0 mg/kg or at 3.0 mg/kg body weight (e.g. in WO2019/008077A1, which describes the use of angiotensin II receptor antagonists for the treatment of systemic diseases and hypertension in cats).

However, according to the present invention it has been found that a preventive effect for the delay of onset of hypertension in cats can be achieved with a lower dosage of the angiotensin II receptor 1 (AT-1) antagonist in a sub-therapeutic range. More specifically, a dosage of the angiotensin II receptor 1 (AT-1) antagonist telmisartan of between 0.50 and 1.0 mg/kg of body weight, preferably of about 1.0 mg/kg of body weight showed an effect to efficiently delay the onset of hypertension in cats. Telmisartan may be administered once, twice or three times a day in a daily dosage as mentioned above.

Thus, according to another aspect, the present invention relates to a method for the prophylaxis of a systemic disease in cats, preferably of chronic kidney disease, e.g. chronic renal failure, wherein the method comprising administration of an effective amount of angiotensin II receptor 1 (AT-1) antagonist (sartan), preferably telmisartan or pharmaceutically acceptable salt thereof, to that cat in need thereof, wherein the effective amount of such angiotensin II receptor 1 (AT-1) antagonist is 0.50 to 1.0 mg/kg of body weight, preferably about 1.0 mg/kg of body weight. Telmisartan may be administered once, twice or three times a day in a daily dosage as mentioned above. In a more specific configuration, the cat is normotensive (SBP of <140 mmHg).

More specifically, the present invention refers to an angiotensin II receptor 1 (AT-1) antagonist (sartan) for use in a method for the prophylaxis of hypertension in a cat in need of such prophylaxis, wherein the method comprises administration of an effective amount of angiotensin II receptor 1 (AT-1) antagonist (sartan) to the cat, wherein the prophylaxis of hypertension is a delay of onset of hypertension and wherein the effective amount of the angiotensin II receptor 1 (AT-1) antagonist administered by oral route is 0.50 to 1.0 mg angiotensin II receptor 1 (AT-1) antagonist per kg of body weight, preferably about 1.0 mg/kg of body weight. In a more specific configuration, the cat is normotensive (SBP of <140 mmHg).

Preferably, said effective amount of such angiotensin II receptor 1 (AT-1) antagonist is between 0.80 and 1.0 mg/kg body weight, more preferably about 1 mg/kg of body weight. The angiotensin II receptor 1 (AT-1) antagonist, preferably telmisartan may be administered once, twice or thrice a day in a daily dosage as mentioned above. It is preferred, when angiotensin II receptor 1 (AT-1) antagonist, preferably telmisartan is administered by oral route a dosage of 0.50 to 1 mg/kg of body weight. Telmisartan may be administered once, twice or three times a day in a daily dosage as mentioned above. In a more specific configuration, the cat is normotensive (SBP of <140 mmHg).

In another embodiment, the present invention relates to a method for the prophylaxis of systemic hypertension (also referred to herein as hypertension) in cats, where the method comprises administration of an effective amount of angiotensin II receptor 1 (AT-1) antagonist (sartan) to a cat in need thereof, where the hypertension is associated with one or more other systemic diseases including (without limitation) CKD, hyperthyroidism, controlled hypertension and idiopathic hypertension. In a further embodiment, the effective amount of the sartan is administered to a cat in a daily dosage amount that is varied over an administration period. The variation of the administration period can be a daily dosage amount of the sartan for a first period of time during the administration period that is 0.5 to 1.0 mg/kg of body weight, and the daily dosage amount of the sartan is decreased for a second period of time subsequent the first period of time during the administration period. The daily dosage amount of the sartan can be decreased for the second period of time by an incremental amount ranging from 0.10 to 0.50 mg/kg of body weight. In an embodiment, the daily dosage amount of the sartan can be decreased for the second period of time by an incremental amount ranging from 0.10 to 0.50 mg/kg of the cat's body weight. The daily dosage amount of the sartan can be decreased based upon a certain criteria for the cat, such as a measured systolic blood pressure (SBP) of the cat. For example, the daily dosage amount of the sartan can be decreased when a SBP value measured for the cat decreases by at least 10 mmHg in relation to a baseline SBP value measured for the cat prior to the first period of time. In another embodiment, the daily dosage amount of the sartan can be decreased when a SBP value measured for the cat is no greater than a predetermined or threshold value (e.g., 120 mmHg). As described herein, a daily dosage amount of sartan can be further decreased in any suitable amounts for any number of consecutive dosage periods and based upon a positive response by the cat to such decreased dosage.

Optionally, the angiotensin II receptor 1 (AT-1) antagonist (sartan), preferably telmisartan can be administered in combination with other drugs. Such other drugs are, for example Ca-channel blockers (e.g. Amlodipine), beta-blockers (e.g. Atenolol, Carvediol), cardiotonic-Ca-sensitising agents (e.g. Pimobendan, Levosimendan), selective If-current inhibitors (i.e. Cilobradine, Ivabradine), ACE inhibitors (e.g. ramipril, benazepril, enalapril); anti-obesity drugs (such as Amphetamine derivatives, Sibutramine, Orlistat, Rimonabat) and the like. Thus, according to another aspect, the present invention relates to a method for the prophylaxis of a systemic disease in cats, preferably of chronic kidney disease, e.g. chronic renal failure, wherein the method comprising administration of an effective amount of angiotensin II receptor 1 (AT-1) antagonist (sartan), preferably telmisartan or pharmaceutically acceptable salt thereof, together with another active substance, to that cat in need of such a treatment, wherein said further active substance is a Ca-channel blocker (e.g. Amlodipine), beta-blocker (e.g. Atenolol, Carvediol), cardiotonic-Ca-sensitising agent (e.g. Pimobendan, Levosimendan), selective If-current inhibitor (i.e. Cilobradine, Ivabradine), ACE inhibitor (e.g. ramipril, benazepril, enalapril); an anti-obesity drug (such as Amphetamine derivatives, Sibutramine, Orlistat, Rimonabat) and the like.

Telmisartan and the other active compounds can be orally administered in a wide variety of different dosage forms, i.e., they may be formulated with various pharmaceutically acceptable inert carriers in the form of tablets, capsules, lozenges, troches, hard candies, powders, sprays, aqueous suspensions, elixirs, syrups, and the like. Such carriers include solid diluents or fillers, sterile aqueous media and various non-toxic organic solvents. Moreover, such oral pharmaceutical formulations can be suitably sweetened and/or flavored by means of various agents of the type commonly employed for such purposes. In general, the compounds of this invention are present in such oral dosage forms at concentration levels ranging from 0.5% to 90% by weight of the total composition, in amounts which are sufficient to provide the desired unit dosages. Other suitable dosage forms for the compounds of this invention include controlled release formulations and devices well known to those who practice in the art.

For purposes of oral administration, tablets containing various excipients such as sodium citrate, calcium carbonate and calcium phosphate may be employed along with various disintegrants such as starch and preferably potato or tapioca starch, alginic acid and certain complex silicate, together with binding agents such as polyvinylpyrrolidone, sucrose, gelatin and acacia. Additionally, lubricating agents such as magnesium stearate, sodium lauryl sulfate and talc or compositions of a similar type may also be employed as fillers in soft and hard-filled gelatin capsules; included lactose or milk sugar as well as high molecular weight polyethylene glycols. When aqueous suspensions and/or elixirs are desired for oral administration, the essential active ingredient therein may be combined with various sweetening or flavoring agents, coloring matter or dyes and, if so desired, emulsifying agents and/or water, ethanol, propylene glycol, glycerin and various like combinations thereof.

For purposes of parenteral administration, solutions of the compounds in sesame or peanut oil or in aqueous propylene glycol may be employed, as well as sterile aqueous solutions of the corresponding pharmaceutically acceptable salts. Such aqueous solutions should be suitably buffered if necessary, and the liquid diluent rendered isotonic with sufficient saline or glucose. These particular aqueous solutions are especially suitable for intravenous, intramuscular and subcutaneous injection purposes. In this connection, the sterile aqueous media employed are readily obtained by standard techniques well known to those skilled in the art. For instance, distilled water is ordinarily used as the liquid diluent and the final preparation is passed through a suitable bacterial filter such as a sintered glass filter or a diatomaceous earth or unglazed porcelain filter. Preferred filters of this type include the Berkefeld, the Chamberland and the Asbestos Disk-Metal Seitz filter, wherein the fluid is sucked into a sterile container with the aid of a suction pump. The necessary steps should be taken throughout the preparation of these inject-able solutions to insure that the final products are obtained in a sterile condition.

For purposes of transdermal administration, the dosage form of the particular compound or compounds may include, by way of example, solutions, lotions, ointments, creams, gels, suppositories, rate-limiting sustained release formulations and devices therefore. Such dosage forms comprise the particular compound or compounds and may include ethanol, water, penetration enhancer and inert carriers such as gel-producing materials, mineral oil, emulsifying agents, benzyl alcohol and the like.

These preformulated combinations of active substances are generally incorporated with one or more formulation adjuvants such as mannitol, sorbitol, xylitol, saccharose, calcium carbonate, calcium phosphate, lactose, croscarmellose sodium salt (cellulose carboxymethylether sodium salt, cross-linked), crospovidone, sodium starch glycolate, hydroxypropylcellulose (low-substituted), maize starch, polyvinylpyrrolidone, copolymers of vinylpyrrolidone with other vinyl derivatives (copovidone), hydroxypropylcellulose, hydroxypropylmethylcellulose, microcrystalline cellulose or starch, magnesium stearate, sodium stearylfumarate, talc, hydroxypropylmethylcellulose, carboxymethylcellulose, cellulose acetate phthalate, polyvinyl acetate, water, water/ethanol, water/glycerol, water/sorbitol, water/polyethyleneglycol, propyleneglycol, cetylstearyl alcohol, carboxy-methylcellulose or fatty substances such as hard fat or suitable mixtures thereof, into conventional galenic preparations such as plain or coated tablets, capsules, powders, suspensions or suppositories.

Tablets may be obtained for example by mixing the active substance or substances with one or more excipients and subsequently compressing them. The tablets may also consist of several layers. Examples of excipients are:
inert diluents such as mannitol, sorbitol, xylitol, saccharose, calcium carbonate, calcium phosphate and lactose;
disintegrants such as croscarmellose sodium salt (cellulose carboxymethylether sodium salt, cross-linked), crospovidone, sodium starch glycolate, hydroxypropylcellulose (low-substituted) and maize starch;
binders such as polyvinylpyrrolidone, copolymers of vinylpyrrolidone with other vinyl derivatives (copovidone), hydroxypropylcellulose, hydroxypropylmethylcellulose, microcrystalline cellulose or starch;
lubricants such as magnesium stearate, sodium stearyl fumarate and talc;
agents for achieving delayed release such as
   hydroxypropylmethylcellulose, carboxymethylcellulose, cellulose acetate phthalate and polyvinyl acetate; and
pharmaceutically permitted colourings such as coloured iron oxides.

Furthermore, if telmisartan is used in combination with another drug used for the prophylaxis of hypertension in cats, the pharmaceutical composition according to the invention may be a kit of parts which comprises:
a first containment containing a pharmaceutical composition comprising an effective amount of telmisartan or a physiologically acceptable salt thereof and one or more pharmaceutically acceptable diluents and/or carriers; and
a second containment containing another drug used for the prophylaxis of a systemic disease, preferably chronic renal failure, or a physiologically acceptable salt thereof and one or more pharmaceutically acceptable diluents and/or carriers.

A preferred kit of parts comprises one or more Ca-channel blockers (e.g. Amlodipine), beta-blockers (e.g. Atenolol, Carvediol), cardiotonic-Ca-sensitising agents (e.g. Pimobendan, Levosimendan), selective If-current inhibitors (i.e. Cilobradine, Ivabradine), ACE inhibitors (e.g. ramipril, benazepril, enalapril); anti-obesity drugs (such as Amphetamine derivatives, Sibutramine, Orlistat, Rimonabat) and the like, in the second containment.

According to a further aspect, the present invention also relates to the use of an angiotensin II receptor 1 (AT-1) antagonist (sartan), preferably of telmisartan for the manufacture of a pharmaceutical composition comprising an effective amount of said angiotensin II receptor 1 (AT-1) antagonist for the prophylaxis of a systemic disease in cats, wherein the prophylaxis is a delay of onset of hypertension.

According to a further aspect, the present invention also relates to the use of an angiotensin II receptor 1 (AT-1) antagonist (sartan), preferably of telmisartan for the manufacture of a pharmaceutical composition comprising an effective amount of said angiotensin II receptor 1 (AT-1) antagonist for the prophylaxis of a systemic disease in cats, wherein the cat has a risk factor for hypertension and wherein the prophylaxis is a delay of onset of hypertension.

According to a further aspect, the present invention also relates to the use of an angiotensin II receptor 1 (AT-1) antagonist (sartan), preferably of telmisartan for the manufacture of a pharmaceutical composition comprising an effective amount of said angiotensin II receptor 1 (AT-1) antagonist for the prophylaxis of hypertension in cats, wherein the cat has a risk factor for hypertension and wherein the prophylaxis of hypertension is a delay of onset of hypertension.

Preferably the systemic disease is selected from the group of cardiovascular diseases, such as dilated cardiomyopathy (DCM), mitral valve insufficiency (MI), hypertrophic cardiomyopathy (HCM) and other acquired or hereditary heart diseases, systemic hypertension, for example hypertension associated with renal diseases, chronic kidney disease (CKD) and other vascular diseases, metabolic disorders like diabetes mellitus. For example, the systemic disease is systemic hypertension associated with one or more of CKD, hyperthyroidism, controlled hypertension and idiopathic hypertension.

Preferred sartans are those mentioned in an exemplarily manner supra. Most preferred is the use of telmisartan or any pharmaceutically acceptable salt thereof, such as Micardis® or Semintra®. The preferred doses which can be used according to the invention are those mentioned supra. Preferred administration routes are orally, bucally, parenterally, nasally, rectally or topically, whereas the oral administration being most preferred. Parenteral administration may include subcutaneous, intravenous, intramuscular and intrasternal injections and infusion techniques.

In another embodiment of the invention, administration of a dosage of a sartan (e.g., telmisartan) is regulated during treatment of a cat and based upon a condition associated with the cat. The dosage regulation can be modified after certain administration periods and in dependence upon a measured change (e.g., improvement) in a physical condition of a cat. For example, in the prophylaxis of hypertension (e.g., hypertension associated with one or more of CKD, hyperthyroidism, controlled hypertension and idiopathic hypertension) in a cat, a sartan can be administered to the cat at an initial dosage amount (e.g., any of the suitable dosage amounts as previously described herein) and then modified after a certain time period, where the modification in dosage amount can be based upon the measured systolic blood pressure (SBP) of the cat. The SBP of the cat can be measured in any suitable manner (e.g., utilizing a blood pressure cuff wrapped around the tail or a limb of the cat). In example embodiments (e.g., such as certain examples described herein), an initial daily dosage amount can be administered to a cat for a certain time period, where the SBP of the cat is determined after such time period. In the event the determined SBP of the cat is not greater than a threshold value, the daily dosage amount can be reduced or titrated down to a smaller amount. Further reductions or down titrations of the daily dosage amount can be implemented in the dosage regimen in the event the SBP of the cat remains below the threshold value and/or drops even further below the threshold value. Alternatively, in other embodiments, the daily dosage amount can also be increased or titrated up in the event the SBP determined for a cat is at a value that exceeds the threshold value by a predetermined amount. The dosages can be administered in any suitable manner such as those previously described herein (e.g., orally, parenterally, etc.) and can further be in any suitable form (e.g., solid or liquid) and combined with any other suitable active ingredient or any other excipient or other substance as previously noted herein.

According to a further aspect of the present invention the present invention relates to an angiotensin II receptor 1 (AT-1) antagonist (sartan), preferably telmisartan or a pharmaceutically acceptable salt thereof, for the use in a method for the prophylaxis of hypertension in a cat, wherein the cat has a risk factor for hypertension and the prophylaxis of hypertension is a delay of onset of hypertension, and wherein the effective amount of the sartan is administered in a daily dosage amount that is varied over an administration period, the daily dosage amount of the sartan for a first period of time during the administration period is 0.5 to 1.0 mg/kg of body weight, and the daily dosage amount of the sartan is decreased for a second period of time subsequent the first period of time during the administration period. In a specific embodiment said hypertension is hypertension which is associated with chronic kidney disease or hyperthyroidism or said hypertension is idiopathic hypertension.

In example embodiment, an initial dosage amount of a sartan can be administered to a cat for prophylaxis of hypertension in an amount of from 0.10 to 1.0 mg/kg, such as 0.20 to 1.0 mg/kg, 0.30 to 1.0 mg/kg, or 0.50 to 1.0 mg/kg (e.g., 0.8 mg/kg or 1.0 mg/kg), where such dosage amount can be administered once per day, twice per day or three times (thrice) per day. A decrease or down titration in daily dosage (as well as an increase or up titration in daily dosage) between a first predetermined or prescribed time period and a second (subsequent) prescribed time period can be in any suitable amount, e.g., a change in daily dosage or incremental change (i.e., an incremental increase or decrease or, in other words, an increase or decrease by a predetermined or stepped amount) in a range of 0.05 to 1.0 mg/kg, or 0.1 to 0.5 mg/kg, etc. For example, changes in daily dosage titration between a first prescribed time period and a second (subsequent) prescribed time period can be modified or adjusted by a delta or incremental (decreased or increased) change of 1.0 mg/kg, 0.9 mg/kg, 0.8 mg/kg, 0.75 mg/kg, 0.5 mg/kg, 0.4 mg/kg, 0.3 mg/kg, 0.25 mg/kg, 0.2 mg/kg, 0.125 mg/kg, 0.1 mg/kg, by 0.05 mg/kg or in any other suitable incremental amount (i.e., any other set decreased or set increased amount).

The daily dosage amount of a sartan administered to a cat can be adjusted over any selected number of time intervals. For example, the initial time period (e.g., a time period including day one of administration) for the initial daily dosage amount of the sartan administered to a cat for the prophylaxis of hypertension can be from 1 day to 30 days or more, such as from 5 days to 28 days, from 7 days to 14 days, etc. At the end of the initial time period, an adjustment that is made in the daily dosage amount can be implemented for a second or subsequent time period that can be from 1 day to 30 days or more, such as from 5 days to 28 days, from 7 days to 14 days, etc. Any further number of subsequent time periods of adjustment in daily dosage can be further implemented as desired and based upon efficacy of administration. In some embodiments, the total administration period at which a cat can be treated with varying daily dosage amounts (e.g., over certain periods of time) can be at least 120 days (e.g., a administration period can last for at least 3 months, at least 4 months, at least 5 months, at least 6 months, etc.).

The methods for prophylaxis of hypertension in cats by administration of a sartan (including modification of dosage of the sartan during an administration period) have been determined to be effective for cats varying in ages. However, no study has been conducted to determine the effectiveness of such methods for a cat having an age of less than 9 months. Accordingly, the methods described herein are preferably for cats having an age of at least 9 months (i.e., an age of 9 months and older). Further, the methods described herein exclude cats having an age of less than 9 months.

As noted herein, for the prophylaxis of hypertension for a cat by administration of a sartan to the cat, the SBP of the cat can be used as the criteria for determining whether an adjustment in the daily dosage to the cat can be implemented. In preferred embodiments, it may be desirable to adjust the daily dosage of the sartan administered to the cat when the SBP of the cat drops below (for down titration or decreased dosage) a first threshold value or rises above (for up titration or increased dosage) a second threshold value. For example, in a scenario in which, after the initial administration period, the SBP of the cat decreases below or is no greater than a first threshold value, the daily dosage amount for administration to the cat can be reduced by any amount (e.g., any of the dosage incremental values as noted herein). An example threshold SBP value can be set, e.g., within a range from 120 mmHg to 170 mmHg. For example, a threshold SBP value can be set at no greater than 170 mmHg, no greater than 165 mmHg, no greater than 160 mmHg, no greater than 155 mmHg, no greater than 150 mmHg, no greater than 145 mmHg, no greater than 140 mmHg, no greater than 135 mmHg, no greater than 130 mmHg, no greater than 125 mmHg, no greater than 120 mmHg or any values there between.

Alternatively, or in combination with establishing a threshold SBP value, a change in SBP value or delta SBP value (where the change or delta is the difference between two SBP values) for the cat can also trigger a modification in the daily dosage amount administered to the cat in another preferred embodiment. For example, a change in SBP value that will trigger a change in dosage amount administered to the cat can be preferably from 10 mmHg to 150 mmHg, 10 mmHg to 100 mmHg, 10 mmHg to 80 mmHg, 10 mmHg to 50 mmHg, 10 mmHg to 30 mmHg, 10 mmHg to 20 mmHg, 20 mmHg to 150 mmHg, 20 mmHg to 100 mmHg, 20 mmHg to 80 mmHg, 20 mmHg to 50 mmHg, 20 mmHg to 30 mmHg, or 5 mmHg to 30 mmHg, such as from 10 mmHg to 25 mmHg, more preferably a change in SBP value of at least 5 mmHg, at least 10 mmHg, at least 15 mmHg, at least 20 mmHg, at least 25 mmHg, or at least 30 mmHg.

In preferred embodiments, the methods of regulation (change in dosage amount) are implemented by administration of the sartan to a cat as a liquid formulation (e.g., any suitable liquid formulation of the types previously described herein). In such embodiments, a suitable dispenser can be provided to facilitate administration at varying dosage amounts. However, the methods of regulation can also be implemented by administration of the sartan as a solid formulation (e.g., as a tablet).

Some preferred non-limiting examples of down titration dosage schemes (which are also described in greater detail in Example A below) are as follows.

A. A sartan (e.g., telmisartan) is administered in liquid form (e.g., using a 10 mg/mL telmisartan solution, and a 2 mL dosing syringe demarcated in 0.10 mL or smaller increments) to a cat for the prophylaxis of hypertension at a daily dosage amount of 1.0 mg/kg (e.g., in two doses per day, each dosage amount being 0.5 mg/kg) for an initial period of 14 days. The daily dosage amount is modified based upon SBP measurements of the cat, e.g., at periodic veterinary visits (e.g., every 2-4 weeks). The cat can have hypertension associated with chronic kidney disease (CKD), hypertension associated with hyperthyroidism, controlled hypertension and/or idiopathic hypertension. Initially (e.g., at or before a first veterinary visit at Day 0), a baseline SBP of the cat is measured. After the initial 14 day period of administration (e.g., at a second veterinary visit, such as at Day 14 of the administration period), the daily dosage amount is decreased or down titrated to 0.5 mg/kg (e.g., in one dose per day). The daily dosage can be further decreased depending upon the SBP value measured for the cat (e.g., at a third visit, such as at Day 28 of the administration period). For example, a decrease in SBP from the baseline SBP value (obtained for the cat prior to the initial period of administration of the sartan) of at least 20 mmHg can result in a decrease in daily dosage. Alternatively (or in combination with a measured decrease in SBP from baseline SBP), a decrease in SBP to a value no greater than a threshold value (where the threshold value is, e.g., 120 mmHg) can result in a decrease in daily dosage. The daily dosage can be decreased by 0.5 mg/kg or less (e.g., 0.5 mg/kg, 0.4 mg/kg, 0.3 mg/kg, 0.25 mg/kg, 0.2 mg/kg, 0.15 mg/kg, 0.125 mg/kg, 0.1 mg/kg or 0.05 mg/kg). Periodically, the dosage can be further decreased in the same or different incremental amounts (e.g., at the second visit, third visit, etc. of the administration period) based upon the cat either maintaining the SBP value at no greater than the threshold value and/or maintaining a decrease in SBP of at least 20 mmHg over a sufficient time period (e.g., 14 days or less). In this example, the initial daily dosage is 1.0 mg/kg, and such daily dosage can be decreased or titrated down (based upon sufficient decrease in SBP) to a daily dosage value ranging from 0.125 mg/kg to 2.0 mg/kg. For example, the daily dosage can be titrated down from 1.0 mg/kg→0.5 mg/kg, etc., where such down titration is implemented when the SBP value of the cat after a certain time period (e.g., 14 days or less) is maintained at no greater than a threshold value (e.g., no greater than 120 mmHg) and/or is maintained at a value lower than at least 20 mmHg from the baseline SBP value for the cat. In a specific example embodiment, the daily dosage amount for the cat can be set at 1.0 mg/kg (e.g., single daily dosage) when the SBP for the cat is maintained within 120-180 mmHg, and is further down titrated (e.g., to 0.5 mg/kg) when the SBP for the cat is maintained at no greater than 120 mmHg. The administration period for the cat can be, e.g., 26 weeks (i.e., 6 months) with visits every 2-4 weeks to determine whether an adjustment is to be made in daily dosage of the sartan based upon the SBP value measured for the cat (e.g., where the daily dosage can be down titrated based upon the SBP value for the cat being maintained at no greater than 120 mmHg).

In certain scenarios, the dosage can be increased or up titrated after having been decreased or down titrated in a previous portion of the administration period if the SBP of the cat has increased to an undesirable value or by an undesirable amount. For example, consider a scenario in which the daily dosage amount of a sartan to a cat was decreased from 1.0 mg/kg daily to 0.5 mg/kg daily (due to a decrease in SBP to no greater than 120 mmHg and/or a decrease from baseline SBP of at least 20 mmHg). In a subsequent time period (e.g., within 14 days of the down titration in dosage), if the SBP of the cat has increased significantly (e.g., the current SBP is 120 mmHg or greater or the current SBP is no longer at least 20 mmHg less than the baseline SBP), the daily dosage of sartan for the cat can be increased or up titrated to a greater value (e.g., the original amount, or to 1.5 mg/kg). During the next period at which the SBP of the cat is measured, an optional further adjustment (decrease or increase) in daily dosage can be implemented depending upon the response of the cat to the change in dosage (based upon the currently measured SBP vs. the previously measured SBP and/or the baseline SBP).

In another aspect, the present invention refers to a method for the delay of onset of hypertension in a cat, wherein the method comprises administration of an effective amount of angiotensin II receptor 1 (AT-1) antagonist (sartan) to a cat.

In another aspect, the present invention refers to a method for the delay of onset of hypertension in a cat with CKD, wherein the method comprises administration of an effective amount of angiotensin II receptor 1 (AT-1) antagonist (sartan) to that cat.

In another aspect, the present invention refers to a method for the prophylaxis of hypertension in a cat, wherein the method comprising administration of an effective amount of angiotensin II receptor 1 (AT-1) antagonist (sartan) to a cat and the prophylaxis of hypertension is a delay of onset of hypertension.

In another aspect, the present invention refers to a method for the treatment or prophylaxis of CKD in a cat, wherein the method comprising administration of an effective amount of angiotensin II receptor 1 (AT-1) antagonist (sartan) to a cat and the treatment or prophylaxis is a delay of onset of hypertension.

In another aspect, the present invention refers to a method for the prophylaxis of hypertension in cats, wherein the method comprising administration of an effective amount of angiotensin II receptor 1 (AT-1) antagonist (sartan) to that cat, wherein the cat has a risk factor for hypertension.

More specifically, in one aspect the method according to the present invention comprises the following steps:

Determination, if the cat has a risk factor for hypertension selected from one or more of the following:
A systolic blood pressure (SBP) between 120 mmHg and 140 mmHg, or between 120 mmHg and 160 mmHg, or between 140 mmHg and 160 mmHg;
Age of the cat;
Hyperthyroidism; or
Chronic Kidney Disease (CKD),
   wherein CKD is preferably characterized by a creatinine value of more than 1.9 mg/dl (168 μmol/L) and less than 5.0 mg/dl (440 μmol/L); and/or
A Urine Specific Gravity (USG) of more than 1.035; and
Administration of an effective amount of angiotensin II receptor 1 (AT-1) antagonist (sartan) to the cat with a risk factor for hypertension.

In a specific configuration, the onset of hypertension is associated with progression of chronic kidney disease or hyperthyroidism. More specifically, the hypertension can be idiopathic hypertension.

In another specific configuration, the angiotensin II receptor 1 (AT-1) antagonist is selected from the group consisting of: candesartan, eprosartan, irbesartan, losartan, tasosartan, telmisartan, valsartan, and a pharmaceutically acceptable salt of said angiotensin II receptor antagonist, preferably telmisartan or a pharmaceutically acceptable salt thereof.

In another embodiment, the effective amount of the angiotensin II receptor 1 (AT-1) antagonist is in a sub-therapeutic range, preferably administered by oral route with 0.50 to 1.0 mg angiotensin II receptor 1 (AT-1) antagonist per kg of body weight.

In another specific embodiment, the effective amount of the sartan is administered in a daily dosage amount that is varied over an administration period, the daily dosage amount of the sartan for a first period of time during the administration period is 0.50 to 1.0 mg/kg of body weight, and the daily dosage amount of the sartan is decreased for a second period of time subsequent the first period of time during the administration period.

In another embodiment, the method further comprises administration of at least one other drug to the cat in need thereof. This drug can be selected from the group consisting of Ca-channel blockers (e.g. Amlodipine), beta-blockers (e.g. Atenolol, Carvediol), cardiotonic-Ca-sensitising agents (e.g. Pimobendan, Levosimendan), selective If-current inhibitors (i.e. Cilobradine, Ivabradine), ACE inhibitors (e.g. ramipril, benazepril, enalapril); anti-obesity drugs (such as Amphetamine derivatives, Sibutramine, Orlistat, Rimonabat).

### EXAMPLES

The following examples serve to further illustrate the present invention; but the same should not be construed as a limitation of the scope of the invention disclosed herein.

### Example 1

The objective of a field trial was to evaluate the efficacy and safety of telmisartan on progression-free survival time of cats with CKD.

This prospective, placebo controlled, randomized, double-blind, multicenter, multinational field study enrolled client-owned cats that were presented in the normal course of veterinary practice to evaluate safety and efficacy of telmisartan on progression-free survival time in cats with CKD compared to placebo. In this study 545 cats were randomly assigned to telmisartan (1 mg/kg body weight) or placebo in a ratio of 1:1. 85% of the cats ranged between 8 and 17 years.

33 cats, which were included in the study had a risk factor for hypertension and were not hypertensive at the beginning of the field study. Those 33 cats, which were included in the field study had a systolic blood pressure (SBP) between 120 mmHg and 160 mmHg. At least two weeks before the start of the field study, creatinine value and Urine Specific Gravity (USG) were determined. Only cats were included in the study which were diagnosed with CKD, meaning a creatinine value ≥1.9 mg/dl (168 μmol/L) and <5.0 mg/dL (440 μmol/L) and a USG of <1.035. On the day of the screening, creatinine and USG value were determined again and cats were excluded, which did not have a creatinine value of ≥1.9 mg/dL (168 μmol/L) and below 5.0 mg/dl (440 μmol/L) or a USG of <1.035.

At all visit timepoints, SBP was recorded prior to performing any other procedures scheduled for the animal during the visit.
5 SBP readings were taken following a ≥10 minute acclimation period.
The highest and the lowest SBP were excluded and a mean of the remaining 3 SBPs was used to obtain the mean SBP for that visit.
   Visits were scheduled on Day 0, 30, 90, 180, 270, 365, 450, 540, 630, 730, 810, 900, 990, 1080, 1170, 1260, 1350, 1440 (each ±7) and, if applicable, additional visits every 3 months (+7 days).

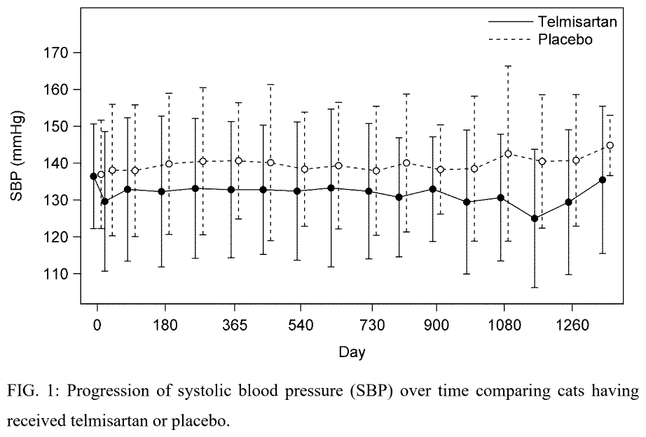

The data (Table 3 and 4), derived from CKD diseased cats, shows that animals having received telmisartan remain non-hypertensive (meaning a SBP below 140 mmHg) for a longer period than cats having received placebo. Table 3 shows the mean probability for the progression of SBP (SBP ≥160 mmHg and an increase in blood pressure of >20 mmHg compared to baseline). Table 4 shows the time to event in days for progression of SBP. In cats having received with telmisartan, the cats reached the endpoint of SBP of 160 mmHg 84.5 days later (median number of days) than in cats having received placebo. FIG. 1 shows the progression of SBP over time comparing cats having received telmisartan or placebo.

Consequently, RAAS inhibition with telmisartan is considered an effective approach to delay the onset of HT and thereby TOD in the feline patients.

TABLE 3
Mean probability for progression of SBP

| | Mean probability for | |
|---|---|---|
| Administration group | progression of SBP | p value |
| Telmisartan | 0.035 | 0.008* |
| Placebo | 0.094 | |

TABLE 4
Time to event in days for progression of SBP (up to 160 mmHg)

| Administration group | No. of animals | Median no. of days |
|---|---|---|
| Telmisartan | 9 | 446.0 |
| Placebo | 24 | 361.5 |

1-11. (canceled)

12. A method for the delay of onset of hypertension in a cat, wherein the method comprises administration of an effective amount of angiotensin II receptor 1 (AT-1) antagonist (sartan) to the cat.

13. A method for the delay of onset of hypertension in a cat with Chronic Kidney Disease (CKD), wherein the method comprises administration of an effective amount of angiotensin II receptor 1 (AT-1) antagonist (sartan) to the cat.

14. A method for the prophylaxis of hypertension in a cat, wherein the method comprises administration of an effective amount of angiotensin II receptor 1 (AT-1) antagonist (sartan) to the cat and the prophylaxis of hypertension is a delay of onset of hypertension.

15. The method according to claim 12, further comprising:
determining whether a risk factor for hypertension in the cat exists based upon one at least one of the following:
a systolic blood pressure (SBP) between 120 mmHg and 140 mmHg, or between 120 mmHg and 160 mmHg, or between 140 mmHg and 160 mmHg;
age of the cat;
hyperthyroidism for the cat; and
Chronic Kidney Disease (CKD) for the cat determined by:
a creatinine value for the cat of more than 1.9 mg/dl (168 μmol/L) and less than 5.0 mg/dl (440 μmol/L), and/or
a Urine Specific Gravity (USG) of more than 1.035; and
administering an effective amount of angiotensin II receptor 1 (AT-1) antagonist (sartan) to the cat with in response to a determinationof a risk factor for hypertension.

16. The method according to claim 12, wherein the delay of onset of hypertension is associated with a slowing down of disease progression of Chronic Kidney Disease (CKD) or hyperthyroidism or a delay of onset of systemic hypertension.

17. The method according to claim 12, wherein the delay of onset of hypertension is idiopathic hypertension.

18. The method according to claim 12, wherein the delay of onset of hypertension is a delay of a time after which a systolic blood pressure of 160 mmHg is reached for the cat compared to cats having not received a sartan.

19. The method according to claim 12, wherein the delay of onset of hypertension is at least 10 days, or at least 20 days, or at least 30 days, or at least 40 days, or at least 50 days, or at least 60 days, or at least 70 days, or at least 80 days compared to cats having not received a sartan.

20. The method according to claim 12, wherein the angiotensin II receptor 1 (AT-1) antagonist is selected from the group consisting of candesartan, eprosartan, irbesartan, losartan, tasosartan, telmisartan, valsartan, and a pharmaceutically acceptable salt of said angiotensin II receptor agonist.

21. The method according to claim 12, wherein the angiotensin II receptor 1 (AT-1) antagonist comprises telmisartan or a pharmaceutically acceptable salt thereof.

22. The method according to claim 12, wherein the angiotensin II receptor 1 (AT-1) antagonist is administered orally.

23. The method according to claim 12, wherein the effective amount of the angiotensin II receptor 1 (AT-1) antagonist is in a sub-therapeutic range.

24. The method according to claim 12, wherein the effective amount of the angiotensin II receptor 1 (AT-1) antagonist is administered in an amount from 0.50 to 1.0 mg/kg body weight of the cat.

25. The method according to claim 12, wherein the effective amount of the angiotensin II receptor 1 (AT-1) antagonist is administered in a daily dosage amount that is varied over an administration period.

26. The method according to claim 25, wherein the daily dosage amount of the angiotensin II receptor 1 (AT-1) antagonist is administered for a first period of time during the administration period from 0.50 to 1.0 mg/kg of body weight, and the daily dosage amount of the angiotensin II receptor 1 (AT-1) antagonist is decreased for a second period of time subsequent the first period of time during the administration period.

27. The method of claim 25, further comprising: reducing the daily dosage of the angiotensin II receptor 1 (AT-1) antagonist over the administration period in response to a measured systolic blood pressure in the cat being maintained at or below a determined level.

28. The method according to claim 12, wherein the pharmaceutical composition comprising the effective amount of angiotensin II receptor 1 (AT-1) antagonist is administered together with at least one other drug to the cat.

29. The method according to claim 28, wherein the other drug is selected from the group consisting of a Ca-channel blocker, a beta-blocker, a cardiotonic-Ca-sensitizing agent, a selective If-current inhibitor, an ACE inhibitors, and an anti-obesity drug.

30. The method according to claim 12, wherein the pharmaceutical composition comprising the effective amount of angiotensin II receptor 1 (AT-1) antagonist is administered together with at least one of Amlodipine, Atenolol, Carvediol, Pimobendan, Levosimendan, Cilobradine, Ivabradine, ramipril, benazepril, enalapril, and an amphetamine derivative.

31. The method according to claim 12, wherein the pharmaceutical composition comprising the effective amount of angiotensin II receptor 1 (AT-1) antagonist is administered together with at least one of Sibutramine, Orlistat, and Rimonabat.
